(19) United States
(12) Patent Application Publication
(10) Pub. No.: US 2017/0281634 A1
(43) Pub. Date: Oct. 5, 2017

(54) MINDFULLNESS-INCREASING COMPOSITION AND METHOD OF USE

(71) Applicant: Ryan Bader, Scottsdale, AZ (US)

(72) Inventor: Ryan Bader, Scottsdale, AZ (US)

(21) Appl. No.: 15/473,419

(22) Filed: Mar. 29, 2017

Related U.S. Application Data
(60) Provisional application No. 62/314,846, filed on Mar. 29, 2016.

(51) Int. Cl. A61K 31/522; A61K 31/405; A61K 31/197

(52) U.S. Cl. CPC A61K 31/522; A61K 31/197; A61K 31/405

(57) ABSTRACT

Disclosed is a mindfulness-increasing composition and method of use. A mixture, some variations of which include 5-amino-3-phenylbuteric acid (phenibut), L-theanine, and 5-hydroxytryptophan (5-HTP), increases focus, mental and physical performance, and calmness while decreasing anxiety and promoting increased mental and physical performance and overall sense of wellbeing through an increased level of mindfulness. The mindfulness-increasing composition also comprises a proprietary neurosupportive blend for optimization of mental and physical performance beneficial to general overall health. An excipient delivery system to provide bioavailability of a palatable, convenient, orally-ingested composition is included. Additionally, methods of use are described, allowing for effective, safe, and convenient use of a mindfulness-increasing composition comprising phenibut.

### CROSS REFERENCE TO RELATED APPLICATION

This application claims priority to United States (Provisional) patent application to Bader, entitled “MINDFULLNESS-INCREASING COMPOSITION AND METHOD OF USE,” application No. 62/314,846, filed Mar. 29, 2016, now pending, the disclosures of which are hereby incorporated entirely herein by reference.

### BACKGROUND OF THE INVENTION

### Technical Field

This invention relates to mindfulness-increasing compositions. In particular, the invention relates to compositions incorporating 4-amino-3-phenybutyric acid, theanine, and 5-hydroxytryptophan in various combinations, methods of formation, and methods of use.

### State of the Art

“Mindfulness” is defined by the Oxford online dictionary as “a mental state achieved by focusing one's awareness on the present moment, while calmly acknowledging and accepting one's feelings, thoughts, and bodily sensations, used as a therapeutic technique. According to the American Psychological Association, mindfulness is a Buddhist concept which arose over 2,000 years ago and involves an expansive moment-to-moment awareness of one's experience without judgement. Recognized benefits of mindfulness include calmness, improved mental clarity and concentration including an increased capacity to focus attention while blocking distracting stimuli, decreased rumination, decreased symptoms of depression, increased working memory capacity, increased positive affect, stress reduction, decrease in anxiety, increased ability to regulate emotions, increased relationship satisfaction, increased attention when performing a specific task, and increased immune function.

Being alert is central to a state of increased mindfulness. For centuries, people have consumed dietary and other supplements to increase and maintain alertness, such as coffee or tea. Although drinks containing natural stimulants, such as the caffeine found in coffee or the theophylline found in tea, for example, are effective in creating an increased state of alertness, such drinks may often increase nervousness or agitation. Such nervousness or agitation represents increased alertness, but may increase distractibility and actually decrease mindfulness. Moreover, such compositions do not create a state of calm.

Accordingly, what is needed is a dietary composition which, unlike simple caffeinated beverages and other currently available compositions, facilitates a state of increased mindfulness in the user.

### DISCLOSURE OF EMBODIMENTS OF THE INVENTION

Embodiments of the present invention include a mindfulness-increasing composition, methods of formation, and methods of increasing mindfulness by using a mindfulness-increasing composition. The foregoing and other features and advantages of the invention will be apparent to those of ordinary skill in the art from the following more particular description of the invention and the accompanying drawings.

Disclosed is a composition comprising 4-amino-3-phenylbutyric acid and L-theonine.

In some embodiments, the composition additionally comprises caffeine. In some embodiments, the composition additionally comprises chamomile flower extract. In some embodiments, the composition additionally comprises lemon balm extract. In some embodiments, the composition additionally comprises a vitamin. In some embodiments, the vitamin is vitamin B3 as niacinamide. In some embodiments, the vitamin is vitamin B6 as pyridoxine hydrochloride. In some embodiments, the vitamin is cyanocobalamin. In some embodiments, the vitamin is vitamin E as alpha tocopherol acetate.

In some embodiments, the composition additionally comprises choline. In some embodiments, the composition additionally comprises inositol. In some embodiments, the composition additionally comprises hyaluronic acid.

Disclosed is a composition comprising 4-amino-3-phenylbutyric acid and 5-hydroxytryptophan.

In some embodiments, the composition additionally comprises caffeine. In some embodiments, the composition additionally comprises chamomile flower extract. In some embodiments, the composition additionally comprises lemon balm extract. In some embodiments, the composition additionally comprises a vitamin. In some embodiments, the vitamin is vitamin B3 as niacinamide. In some embodiments, the vitamin is vitamin B6 as pyridoxine hydrochloride. In some embodiments, the vitamin is cyanocobalamin. In some embodiments, the vitamin is vitamin E as alpha tocopherol acetate.

In some embodiments, the composition additionally comprises choline. In some embodiments, the composition additionally comprises inositol. In some embodiments, the composition additionally comprises hyaluronic acid.

Disclosed is a composition comprising L-theanine and 5-hydroxytryptophan.

In some embodiments, the composition additionally comprises caffeine. In some embodiments, the composition additionally comprises chamomile flower extract. In some embodiments, the composition additionally comprises lemon balm extract. In some embodiments, the composition additionally comprises a vitamin. In some embodiments, the vitamin is vitamin B3 as niacinamide. In some embodiments, the vitamin is vitamin B6 as pyridoxine hydrochloride. In some embodiments, the vitamin is cyanocobalamin. In some embodiments, the vitamin is vitamin E as alpha tocopherol acetate.

In some embodiments, the composition additionally comprises choline. In some embodiments, the composition additionally comprises inositol. In some embodiments, the composition additionally comprises hyaluronic acid.

Disclosed is a composition comprising a mixture of 4-amino-3-hydroxybutyric acid, L-theanine, and 5-hydroxytryptophan in a ratio of about thirteen (13) parts 4-amino-3-hydroxybutyric acid to about four and one half (4.5) parts L-theanine to about one part 5-hydroxytryptophan.

In some embodiments, the mixture is dissolved in water at a total concentration of about twenty-three point five (23.5) parts by weight of mixture to one (1) part by weight of water to form an aqueous solution.

In some embodiments, the composition additionally comprises caffeine. In some embodiments, the composition additionally comprises chamomile flower extract. In some embodiments, the composition additionally comprises lemon balm extract. In some embodiments, the composition additionally comprises a vitamin. In some embodiments, the vitamin is vitamin B3 as niacinamide. In some embodiments, the vitamin is vitamin B6 as pyridoxine hydrochloride. In some embodiments, the vitamin is cyanocobalamin. In some embodiments, the vitamin is vitamin E as alpha tocopherol acetate.

Disclosed is a serving of a mindfulness-increasing composition comprising 4-amino-3-phenylbutyric acid, about 1,000 mg.; L-theanine, about 335 mg.; lemon balm extract, about 124 mg.; inositol, about 115 mg.; choline, about 115 mg.; chamomile flower extract, about 100 mg.; 5-hydroxy-L-tryptohpan, about 7 mg.; caffeine, about 50 mg.; hyaluronic acid, about 10 mg.; d-alpha tocopherol acetate, about 15 international units (“IU”); niacinamide, about 100 mg.; pyridoxine hydrochloride, about 25 mg.; and cyanocobalamin, about 100 micrograms (“mcg.”) in an aqueous solution wherein the total volume of the serving is about 60 (sixty) milliliters.

In some embodiments, the amount of 4-amino-3-phenylbutyric acid is between about 250 milligrams (“mg”) and about 1,500 mg. In some embodiments, the amount of 4-amino-3-phenylbutyric acid is between about 20 mg and about 300 mg. In some embodiments, the amount of 4-amino-3-phenylbutyric acid is between about 300 mg and about 350 mg. In some embodiments, the amount of 4-amino-3-phenylbutyric acid is between about 350 mg and about 400 mg. In some embodiments, the amount of 4-amino-3-phenylbutyric acid is between about 400 mg and about 450 mg. In some embodiments, the amount of 4-amino-3-phenylbutyric acid is between about 450 mg and about 500 mg. In some embodiments, the amount of 4-amino-3-phenylbutyric acid is between about 550 mg and about 600 mg. In some embodiments, the amount of 4-amino-3-phenylbutyric acid is between about 600 mg and about 650 mg. In some embodiments, the amount of 4-amino-3-phenylbutyric acid is between about 650 mg and about 700 mg. In some embodiments, the amount of 4-amino-3-phenylbutyric acid is between about 700 mg and about 750 mg. In some embodiments, the amount of 4-amino-3-phenylbutyric acid is between about 750 mg and about 800 mg. In some embodiments, the amount of 4-amino-3-phenylbutyric acid is between about 380 mg and about 850 mg. In some embodiments, the amount of 4-amino-3-phenylbutyric acid is between about 850 mg and about 900 mg. In some embodiments, the amount of 4-amino-3-phenylbutyric acid is between about 900 mg and about 950 mg. In some embodiments, the amount of 4-amino-3-phenylbutyric acid is between about 950 mg and about 1,000 mg. In some embodiments, the amount of 4-amino-3-phenylbutyric acid is between about 1,000 mg and about 1,100 mg. In some embodiments, the amount of 4-amino-3-phenylbutyric acid is between about 1,100 mg and about 1,200 mg. In some embodiments, the amount of 4-amino-3-phenylbutyric acid is between about 1,200 mg and about 1,300 mg. In some embodiments, the amount of 4-amino-3-phenylbutyric acid is between about 1,300 mg and about 1,400 mg. In some embodiments, the amount of 4-amino-3-phenylbutyric acid is between about 1,400 mg and about 1,500 mg. In some embodiments, the amount of 4-amino-3-phenylbutyric acid is greater than about 1,500 mg. In some embodiments, the amount of 4-amino-3-phenylbutyric acid is less than about 250 mg.

In some embodiments, the amount of L-theanine is between about 50 mg and about 400 mg. In some embodiments, the amount of L-theanine is between about 50 mg and about 100 mg. In some embodiments, the amount of L-theanine is between about 100 mg and about 150 mg. In some embodiments, the amount of L-theanine is between about 150 mg and about 200 mg. In some embodiments, the amount of L-theanine is between about 200 mg and about 250 mg. In some embodiments, the amount of L-theanine is between about 250 mg and about 300 mg. In some embodiments, the amount of L-theanine is between about 300 mg and about 350 mg. In some embodiments, the amount of L-theanine is between about 350 mg and about 400 mg. In some embodiments, the amount of L-theanine is greater than about 400 mg. In some embodiments, the amount of L-theanine is less than about 50 mg.

In some embodiments, the amount of 5-hydroxytryptophan is between about 50 mg and about 300 mg. In some embodiments, the amount of 5-hydroxytryptophan is between about 50 mg and about 100 mg. In some embodiments, the amount of 5-hydroxytryptophan is between about 150 mg and about 200 mg. In some embodiments, the amount of 5-hydroxytryptophan is between about 200 mg and about 250 mg. In some embodiments, the amount of 5-hydroxytryptophan is between about 250 mg and about 300 mg. In some embodiments, the amount of 5-hydroxytryptophan is greater than about 300 mg. In some embodiments, the amount of 5-hydroxytryptophan is less than about 50 mg.

In some embodiments, the amount of caffeine is between about 50 mg and about 400 mg. In some embodiments, the amount of caffeine is about 50 mg. In some embodiments, the amount of caffeine is between about 50 mg and about 100 mg. In some embodiments, the amount of caffeine is between about 100 mg and about 150 mg. In some embodiments, the amount of caffeine is between about 150 mg and about 200 mg. In some embodiments, the amount of caffeine is between about 200 mg and about 250 mg. In some embodiments, the amount of caffeine is between about 250 mg and about 300 mg. In some embodiments, the amount of caffeine is between about 300 mg and about 350 mg. In some embodiments, the amount of caffeine is between about 350 mg and about 400 mg. In some embodiments, the amount of caffeine is greater than about 400 mg. In some embodiments, the amount of caffeine is less than about 50 mg.

In some embodiments, and amount of chamomile flower extract is between about 400 mg. and about 1,600 mg. In some embodiments, and amount of chamomile flower extract is between about 400 mg. and about 500 mg. In some embodiments, and amount of chamomile flower extract is between about 500 mg. and about 600 mg. In some embodiments, and amount of chamomile flower extract is between about 600 mg. and about 700 mg. In some embodiments, and amount of chamomile flower extract is between about 700 mg. and about 800 mg. In some embodiments, and amount of chamomile flower extract is between about 800 mg. and about 900 mg. In some embodiments, and amount of chamomile flower extract is between about 900 mg. and about 1,000 mg. In some embodiments, and amount of chamomile flower extract is between about 1,000 mg. and about 1,100 mg. In some embodiments, and amount of chamomile flower extract is between about 1,100 mg. and about 1,200 mg. In some embodiments, and amount of chamomile flower extract is between about 1,200 mg. and about 1,300 mg. In some embodiments, and amount of chamomile flower extract is between about 1,300 mg. and about 1,400 mg. In some embodiments, and amount of chamomile flower extract is between about 1,400 mg. and about 1,500 mg. In some embodiments, and amount of chamomile flower extract is between about 1,500 mg. and about 1,600 mg. In some embodiments, and amount of chamomile flower extract is greater than about 1,600 mg. In some embodiments, the amount of chamomile flower extract is less than about 400 mg.

In some embodiments, the amount of lemon balm extract is between about 600 mg and about 1,600 mg. In some embodiments, and amount of lemon balm extract is between about 600 mg and about 700 mg. In some embodiments, and amount of lemon balm extract is between about 700 mg and about 800 mg. In some embodiments, and amount of lemon balm extract is between about 800 mg and about 900 mg. In some embodiments, and amount of lemon balm extract is between about 900 mg and about 1,000 mg. In some embodiments, and amount of lemon balm extract is between about 1,000 mg and about 1,100 mg. In some embodiments, and amount of lemon balm extract is between about 1,100 mg and about 1,200 mg. In some embodiments, and amount of lemon balm extract is between about 1,200 mg and about 1,300 mg. In some embodiments, and amount of lemon balm extract is between about 1,300 mg and about 1,400 mg. In some embodiments, and amount of lemon balm extract is between about 1,400 mg and about 1,500 mg. In some embodiments, and amount of lemon balm extract is between about 1,500 mg and about 1,600 mg. In some embodiments, and amount of lemon balm extract is greater than about 1,600 mg. In some embodiments, and amount of lemon balm extract is less than about 600 mg.

In some embodiments, the amount of pyridoxine hydrochloride is between about 5 mg and about 50 mg. In some embodiments, the amount of pyridoxine hydrochloride is between about 5 mg and about 10 mg. In some embodiments, the amount of pyridoxine hydrochloride is between about 10 mg and about 15 mg. In some embodiments, the amount of pyridoxine hydrochloride is between about 15 mg and about 20 mg. In some embodiments, the amount of pyridoxine hydrochloride is between about 20 mg and about 25 mg. In some embodiments, the amount of pyridoxine hydrochloride is between about 25 mg and about 30 mg. In some embodiments, the amount of pyridoxine hydrochloride is between about 30 mg and about 35 mg. In some embodiments, the amount of pyridoxine hydrochloride is between about 35 mg and about 40 mg. In some embodiments, the amount of pyridoxine hydrochloride is between about 40 mg and about 45 mg. In some embodiments, the amount of pyridoxine hydrochloride is between about 45 mg and about 50 mg. In some embodiments, the amount of pyridoxine hydrochloride is greater than about 50 mg. In some embodiments, the amount of pyridoxine hydrochloride is less than about 5 mg.

In some embodiments, the amount of cyanocobalamin is between about 0.05 mg and about 15 mg. In some embodiments, the amount of cyanocobalamin is between about 0.05 mg and about 0.15 mg. In some embodiments, the amount of cyanocobalamin is between about 0.15 mg and about 0.50 mg. In some embodiments, the amount of cyanocobalamin is between about 0.50 mg and about 1.0 mg. In some embodiments, the amount of cyanocobalamin is between about 1.0 mg and about 5.0 mg. In some embodiments, the amount of cyanocobalamin is between about 5.0 mg and about 10.0 mg. In some embodiments, the amount of cyanocobalamin is between about 10.0 mg and about 15.0 mg. In some embodiments, the amount of cyanocobalamin is greater than about 15.0 mg. In some embodiments, the amount of cyanocobalamin is less than about 0.05 mg.

In some embodiments, and amount of choline is between about 50 mg and about 500 mg. In some embodiments, and amount of choline is between about 50 mg and about 100 mg. In some embodiments, and amount of choline is between about 100 mg and about 150 mg. In some embodiments, and amount of choline is between about 150 mg and about 200 mg. In some embodiments, and amount of choline is between about 200 mg and about 250 mg. In some embodiments, and amount of choline is between about 250 mg and about 300 mg. In some embodiments, and amount of choline is between about 300 mg and about 350 mg. In some embodiments, and amount of choline is between about 350 mg and about 400 mg. In some embodiments, and amount of choline is between about 400 mg and about 450 mg. In some embodiments, and amount of choline is between about 450 mg and about 500 mg. In some embodiments, and amount of choline is greater than about 500 mg. In some embodiments, and amount of choline is less than about 50 mg.

In some embodiments, and amount of inositol is between about 50 and about 3000 mg. In some embodiments, and amount of inositol is between about 50 and about 100 mg. In some embodiments, and amount of inositol is between about 100 and about 150 mg. In some embodiments, and amount of inositol is between about 150 and about 200 mg. In some embodiments, and amount of inositol is between about 200 and about 250 mg. In some embodiments, and amount of inositol is between about 250 and about 300 mg. In some embodiments, and amount of inositol is between about 300 and about 400 mg. In some embodiments, and amount of inositol is between about 400 and about 500 mg. In some embodiments, and amount of inositol is between about 500 and about 600 mg. In some embodiments, and amount of inositol is between about 600 and about 800 mg. In some embodiments, and amount of inositol is between about 800 and about 1,000 mg. In some embodiments, and amount of inositol is between about 1,000 and about 1,500 mg. In some embodiments, and amount of inositol is between about 1,500 and about 2,000 mg. In some embodiments, and amount of inositol is between about 2,000 and about 3,000 mg. In some embodiments, and amount of inositol is greater than about 3,000 mg. In some embodiments, and amount of inositol is less than about 50 mg.

In some embodiments, the amount of hyaluronic acid is between about 5 mg. and about 200 mg. In some embodiments, the amount of hyaluronic acid is between about 25 mg. and about 50 mg. In some embodiments, the amount of hyaluronic acid is between about 50 mg. and about 100 mg. In some embodiments, the amount of hyaluronic acid is between about 100 mg. and about 200 mg. In some embodiments, the amount of hyaluronic acid is greater than about 200 mg. In some embodiments, the amount of hyaluronic acid is less than about 5 mg.

In some embodiments, the mindfulness-increasing composition additionally comprises a flavoring agent, sucralose, potassium sorbate; sodium benzoate; malic acid; citric acid; acesulfame potassium, and an artificial color.

Disclosed is a method of forming a mindfulness-increasing composition comprising a providing a mixture comprising 4-amino-3-phenylbutyric acid and L-theanine; and batching the mixture with ingredients of a mindfulness-increasing formulation into a solution.

In some embodiments, the method further comprises a step packaging the mindfulness-increasing composition into a consumer unit.

Disclosed is a method of forming a mindfulness-increasing composition comprising a providing a mixture comprising L-theanine; and 5-hydroxytryptophan; and batching the mixture with ingredients of a mindfulness-increasing formulation into a solution.

In some embodiments, the method further comprises a step packaging the mindfulness-increasing composition into a consumer unit.

Disclosed is a method of forming a mindfulness-increasing composition comprising a providing a mixture comprising 4-amino-3-phenylbutyric acid and 5-hydroxytryptophan; and batching the mixture with ingredients of a mindfulness-increasing formulation into a solution.

In some embodiments, the method further comprises a step packaging the mindfulness-increasing composition into a consumer unit.

### BRIEF DESCRIPTION OF THE DRAWINGS

FIG. 1 is a schematic representation of a mindfulness-increasing composition;

FIG. 2 is a flowchart showing steps of a method 200 for forming a mindfulness-increasing composition;

FIG. 3 is a flowchart showing steps of a method 300 for forming a mindfulness-increasing composition;

FIG. 4 is a flowchart showing steps of a method 400 for forming a mindfulness-increasing composition; and

FIG. 5 is a flowchart showing steps of a method 500 for providing a mindfulness-increasing composition to improve a mental condition.

### DETAILED DESCRIPTION OF EMBODIMENTS OF THE INVENTION

As mentioned herein above, the disclosed invention relates to mindfulness-increasing compositions. In particular, the invention relates to compositions incorporating 4-amino-3-phenybutyric acid, L-theanine, and 5-hydroxytryptophan in various combinations, methods of formation, and methods of use.

Derivatives of the neurotransmitter gamma aminobutyric acid (“GABA”) may compete with the naturally occurring neurotransmitter and bind to GABA receptors in the central nervous system. One such derivative, 4-amino-3-phenylbutyric acid (“phenibut”) comprises GABA to which a phenyl ring has been added to the third carbon atom of the butyric acid chain, enabling phenibut to cross the blood-brain barrier. Phenibut has a tranquilizing effect and reduces anxiety similar to an anxiolytic, particularly social anxiety. Phenibut may, additionally, improve cognitive function.

Theanine is a naturally occurring amino acid analog to L-glutamine and L-glutamate. Theanine increases serotonin, dopamine, gamma-aminobutyric acid (“GABA”), and glycine levels in multiple areas of the brain, and L-Theanine may increase mental focus and improve concentration while exerting an overall calming effect. Other effects of L-theanine may include stress reduction, elevation of mood, and increased cognitive performance. Theanine is additionally able to cross the blood-brain barrier and has reported psychoactive properties. Theanine has been studied for its potential ability to reduce mental and physical stress, improve cognition, and boost mood and cognitive performance in a synergistic manner with caffeine.

Another naturally occurring amino acid, 5-hydroxytryptophan (“5-HTP”), is a metabolic intermediate in in the synthesis of serotonin and melatonin from the amino acid tryptophan. Serotonin is a neurotransmitter. Melatonin acts as a hormonal substance and is involved in regulation of the wake-sleep cycles in mammals, including humans. 5-HTP may elevate mood, suppress the appetite, and promote sleep in persons with insomnia. In addition to insomnia, 5-HTP may also be useful in treating depression, anxiety, migraine and tension-type headaches, fibromyalgia, binge eating associated with obesity, premenstrual syndrome, premenstrual dysphoric disorder, and attention deficit-hyperactivity disorder.

Some embodiments of the disclosed invention incorporate combinations of phenibut, L-theanine, and 5-HTP. Ingestion of a composition comprising safe, non-toxic amounts of these compounds, in different combinations, with or without the addition of other compounds, increases mindfulness through synergistically increasing alertness and focus while exerting a calming, relaxing, and mood-elevating effect. Phenibut, L-theanine, and 5-HTP may act in combination synergistically, potentiating the effects of GABA and serotonin in the brain, resulting in an increased awareness accompanied by decreased anxiety and an increased sense of focus and calm.

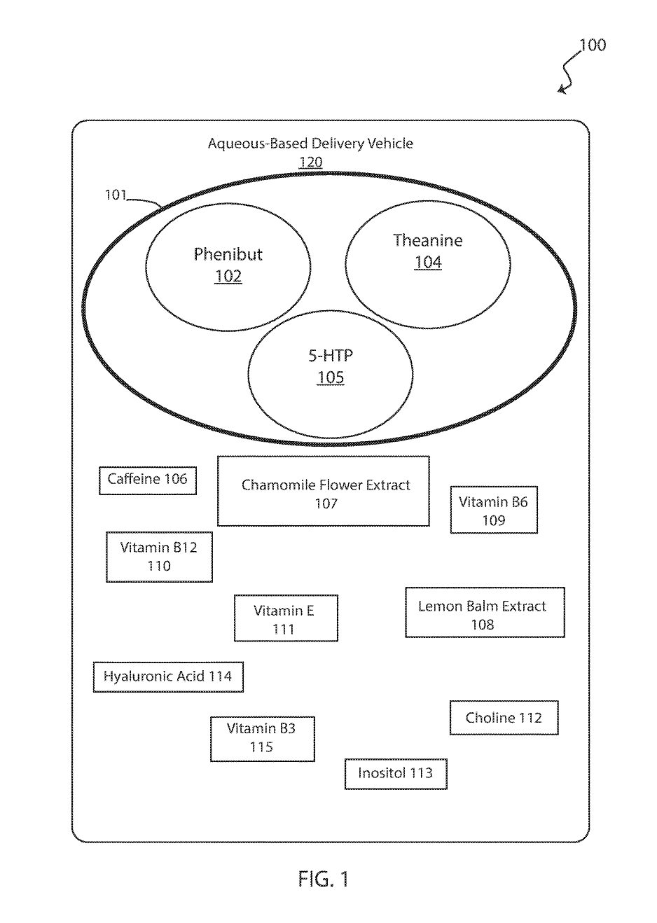

FIG. 1 is a schematic representation of a mindfulness-increasing composition 100. Composition 100, as shown by FIG. 1, comprises a phenibut 102. In some embodiments, composition 100 additionally comprises a theanine 104. In some embodiments, theanine 104 is the L-enantiomer of theanine. In some embodiments, theanine 104 is a racemic mixture of theanine. In some embodiments, composition 100 additionally comprises a 5-HTP 105. In some embodiments, composition 100 comprises theanine 104 and 5-HTP 105 and does not comprise phenibut 102.

FIG. 1 also shows a mixture 101. In some embodiments, mixture 101 comprises phenibut 102, theanine 104, and 5-HTP 105. When ingested, mixture 101 increases focus, relieves anxiety, increases calm, and creates a general sense of wellbeing to a greater degree than expected by separately consuming these ingredients. It is possible that phenibut 102, theanine 104, and 5-HTP 105 within mixture 101 act within the central nervous system to potentiate the effects of one another, possibly through GABA and 5-hydroxytryptamine receptor regulation. In some embodiments, mixture 101 comprises about 1000 mg. of phenibut 102, about 335 mg. of theanine 104 wherein the theanine 104 comprises primarily L-theanine, and about 75 mg. of 5-HTP 105 to form a single serving of mixture 101. In some embodiments, mixture 101 comprises phenibut 102, theanine 104, and 5-HTP 105 in a ratio of about 13 parts phenibut 102 to about 4.5 parts theanine 104 to about 1 part 5-HTP. In some embodiments, mixture 101 is dissolved in water to form an aqueous solution with a total volume of about 60 ml. In some embodiments, mixture 101 is dissolved in water to form an aqueous solution wherein the mixture is at a concentration of about 23.3 parts by weight of mixture 101 to about 1 part by weight of water.

Additional compounds are mixed with phenibut 102, theanine 104, and 5-HTP 105 in some embodiments of the invention to potentiate the effects of phenibut 102, theanine 104, and 5-HTP 105. For example, in some embodiments, composition 100 additionally comprises a caffeine 106. Caffeine 106 acts in composition to synergistically augment the central nervous system stimulant effects of theanine. Additionally, caffeine 106 reduces or prevents drowsiness and physical fatigue, in some embodiments.

In some embodiments, a chamomile flower extract 107 is combined with phenibut 102 in composition 100. Chamomile flower extract 107 may potentiate the anxiolytic properties of phenibut, and is incorporated into some embodiments of composition 100 to reduce or prevent stress, and to prevent or reduce sleeplessness or insomnia. In addition to chamomile flower extract 107, in some embodiments, composition 100 comprises a lemon balm extract 108. In some embodiments, composition 100 comprises lemon balm extract 108 but does not comprise chamomile flower extract 107. Lemon balm extract 108 is through to potentiate the effects of phenibut 102 (and chamomile flower extract 107) in decreasing or preventing stress and insomnia. Additionally, lemon balm extract 108, in some embodiments, may potentiate the anxiolytic properties of phenibut 102.

Some embodiments of composition 100 additionally comprise various combinations of B-vitamins. For example, in some embodiments, composition 100 comprises a vitamin B3 115, as niacinamide. Vitamin B3 115 has a wide range of effects in the body, promoting digestive, integumentary, and nervous system health. Composition 100 comprises a vitamin B12 110, as cyanocobalamin, in some embodiments, Vitamin B12 110 is necessary for red blood cell production and nervous system function. A vitamin B6 109, as pyridoxine, is present in some embodiments of composition 100. Vitamin B6 109 promotes healthy central and peripheral nervous system function and also promotes immune system function and antibody production.

Some embodiments of composition 100 additionally comprise a vitamin E 111, as the acetate salt of d-alpha-tocopherol. Vitamin E 111, in some embodiments, provides composition 100 with antioxidant properties to increase overall cellular health. Vitamin E 111 additionally adds to the tendency of composition 100 to enhance immune function. Some embodiments of composition 100 additionally comprise a choline 112. Choline 112 supports hepatocyte function, normal central nervous system development, neve function, muscle contraction energy levels, and regulating metabolism at a healthy level. In some embodiments of composition 100 comprising choline 112, choline 112 may promote passage of phenibut 102, theanine 104, and 5-HTP 105 across the blood-brain barrier. Some embodiments of composition 100 additionally comprise an inositol 113. Inositol 113 is a naturally occurring compound with anxiolytic properties. The combination of inositol 113 and phenibut 102 is thought to augment the anti-anxiety properties of phenibut 102 and increase the overall anxiolytic function of composition 100, in some embodiments. Inositol 113 is useful in treating severe forms of anxiety, including panic disorder, obsessive-compulsive disorder, and agoraphobia. Inositol 113 facilitates the activity of neurotransmitters within the brain.

Although not directly related to the positive effects of composition 100 on various mental states, such as focus, anxiety, and alertness, a hyaluronic acid 114 is present in some embodiments of composition 100 to increase a sense of well-being. Hyaluronic acid 114 acts to hydrate and lubricate cartilaginous tissues, wherein hyaluronic acid 114 may promote health joint function and decrease joint pain, when present. Decreased joint pain promotes an overall sense of improved well-being to a user of composition 100 comprising hyaluronic acid 114, in some embodiments.

As additionally shown in FIG. 1, mindfulness-increasing composition comprises an aqueous-based delivery vehicle 120, in some embodiments. One non-limiting example of an aqueous-based delivery vehicle 120 comprises water, a flavor, a sweetener, a caffeine-masker to create a palatable, easily consumed composition for ingestion by the end user. This is not meant to be limiting. There are many flavorings, sweeteners, and additional compounds used in the arts to make aqueous compounds more palatable and easy for a user to ingest and many choices and combinations of flavorings, sweeteners, and additional compounds are possible. A non-limiting example of a sweetener found in some embodiments of aqueous-based delivery vehicle 120 is sucralose. A second non-limiting example is acesulfame. Some examples of preservatives include sodium benzoate and potassium sorbate. There are almost limitless examples of natural and artificial flavoring compounds acceptable for incorporation in aqueous-based delivery vehicle 120 of composition 100.

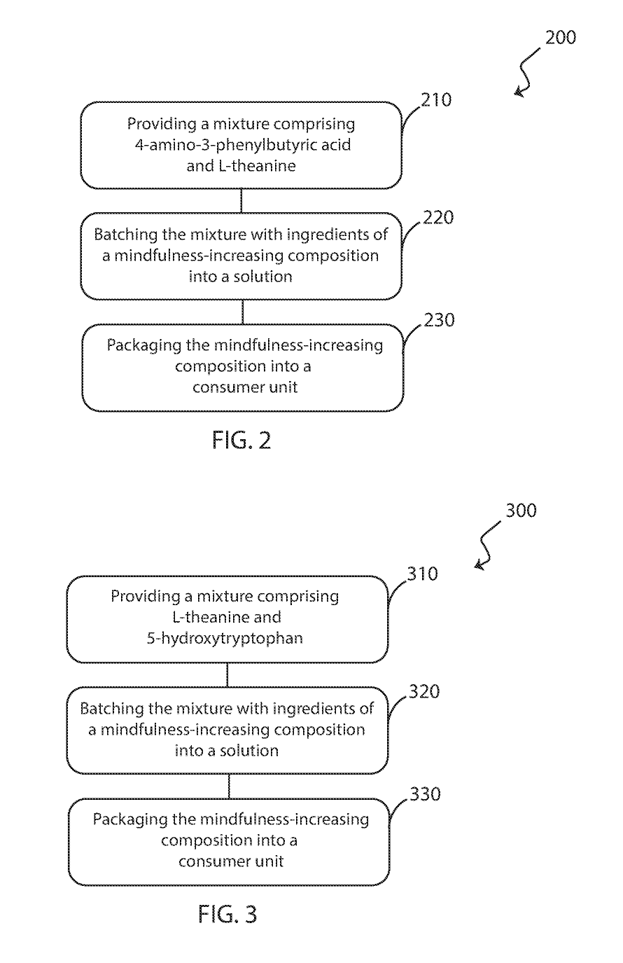

FIG. 2 is a flowchart showing steps of a method 200 for forming a mindfulness-increasing composition. As shown in FIG. 2, method 200 comprises a providing step 210; and a batching step 220.

Providing step 210, in some embodiments, comprises providing a mixture comprising 4-amino-3-phenylbutyric acid and L-theanine. In some embodiments, the mixture additionally comprises 5-HTP.

Batching step 220, in some embodiments, comprises batching the mixture with ingredients of a mindfulness-increasing composition into solution. The ingredients include the mixture comprising 4-amino-3-phnylbutyric acid and L-theanine from providing step 210 described herein above. In some embodiments, batching step 210 comprises a single step combining all ingredients together and mixing until the solid ingredients are completely dissolved into solution with the liquid ingredients, primarily water.

In some embodiments, batching step 220 comprises multiple sub-steps (not shown in the FIG. 2) wherein the various ingredients of the mindfulness-increasing composition are combined. In one non-limiting example, in some embodiments, hot water, at a temperature of about 140 degrees Fahrenheit, is added to a tank-like vessel containing preservatives and mixed until the preservatives are dissolved in the hot water. In some embodiments, the preservatives comprise potassium sorbate and sodium benzoate. The aforementioned preservatives are given by example only and are not meant to be limiting. To the hot water-preservative solution is added sodium hyaluronate and the fluid is mixed for about twenty minutes, or until the sodium hyaluronate is fully dissolved in the hot water-preservative solution, in some embodiments. Next, in some embodiments, a stress-relief energy premix comprising a combination of the mixture with one, some, or all of the following ingredients: lemon balm extract; inositol; choline; chamomile flower extract; caffeine; d-alpha tocopherol acetate; niacinamide; pyridoxine hydrochloride; and cyanocobalamin is combined with the hot water-preservative solution and stirred for about twenty minutes, or until all components of the stress-relief energy premix are dissolved.

In some embodiments, flavorings, including a sweetener, are then added to the solution during batching step 220. In some embodiments, a caffeine masking agent is also added to the solution. Following addition of all ingredients to the solution, and complete dissolution of all solid ingredients into the solution, additional water is added, in some embodiments, to adjust the concentration of one or more than one of the ingredients in the solution. In some embodiments, the resulting solution is then mixed for about an additional twenty minutes. In some embodiments, a final concentration of one or more than one of the ingredients is checked. In some embodiments, the solution is checked for taste.

In some embodiments, method 200 further comprises a packaging step 230, as shown in FIG. 2. Packaging step 230 of method 200 comprises packaging the mindfulness-increasing composition into a consumer unit. In some embodiments, packaging step 230 further comprises filling a screw-top plastic bottle with about sixty (60) milliliters of the mindfulness-increasing composition and screwing the screw top onto the bottle, sealing the consumer unit. In some embodiments, packaging step 230 comprises use of an alternative packaging container, such as a peel-top container, a can, a soft-foil package which the end user punctures to consume the mindfulness-increasing composition within, or the like. In some embodiments, packaging step 220 further comprises affixing a label to the consumer unit. In some embodiments, packaging step 230 further comprises combining a plurality of consumer units into a group package for distribution to a retail outlet or a distributor for online sales to an individual consumer end-user.

FIG. 3 is a flowchart showing steps of a method 300 for forming a mindfulness-increasing composition. As shown in FIG. 3, method 300 comprises a providing step 310; and a batching step 320. In some embodiments, FIG. 3 additionally comprises a packaging step 330.

Providing step 310, in some embodiments, comprises providing a mixture comprising 4-amino-3-phenylbutyric acid and 5-hydroxytryptophan. In some embodiments, the mixture additionally comprises L-Theanine. Batching step 320 is similar to batching step 220 described in detail herein above. Packaging step 330 is similar to packaging step 230 described in detail herein above.

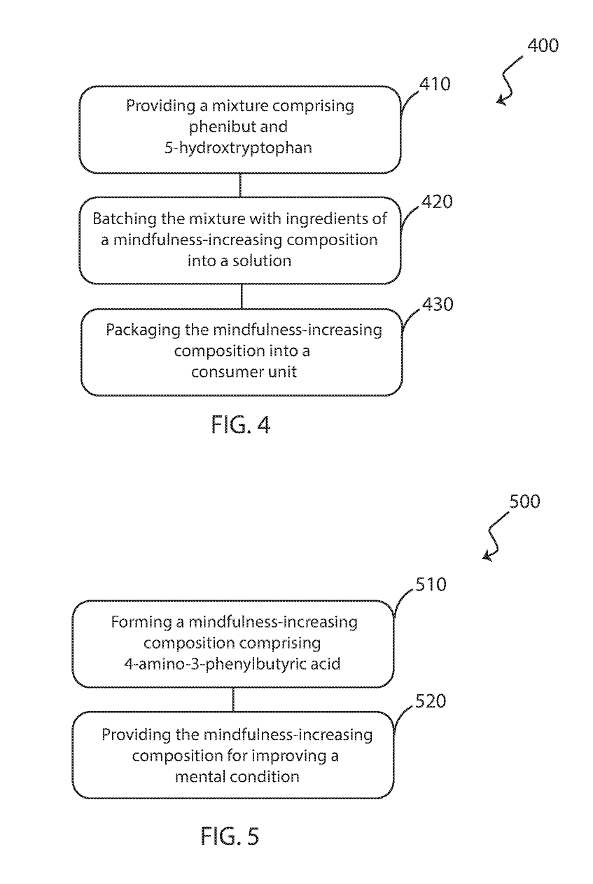

FIG. 4 is a flowchart showing steps of a method 400 for forming a mindfulness-increasing composition. As shown in FIG. 4, method 400 comprises a providing step 410; and a batching step 420. In some embodiments, FIG. 4 additionally comprises a packaging step 330.

Providing step 410, in some embodiments, comprises providing a mixture comprising 4-amino-3-phenylbutyric acid (phenibut) and 5-hydroxytryptophan. In some embodiments, the mixture additionally comprises L-Theanine. Batching step 420 is similar to batching step 220 described in detail herein above. Packaging step 430 is similar to packaging step 230 described in detail herein above.

FIG. 5 shows a flowchart showing steps of a method 500 for providing a mindfulness-increasing composition to facilitate a state of increased mindfulness. Method 300 comprises a forming step 510 and a providing step 520.

Forming step 510, in some embodiments, comprises forming a mindfulness-increasing composition. In some embodiments, forming step 510 comprises steps 210 and 220 of method 200. In some embodiments, forming step 510 comprises steps 310 and 320 of method 300. In some embodiments forming step 520 comprises steps 410 and 420 of method 400.

Providing step 520 comprises providing a mindfulness-increasing composition for improving a mental condition. In some embodiments, the mental condition is stress. The mindfulness-increasing composition is provided for use, in some embodiments, to decrease stress associated with a stressful situation. Some non-limiting examples of stressful situations include traveling, public speaking, daily stress associated with parenting or managing a household, or relief of stress accumulated over the course of a working week prior to relaxing on the weekend.

In some embodiments, the mental condition is a lack of focus. The mindfulness-increasing composition is provided for use, in some embodiments, to increase the degree of mental focus on a particular task. The performance of almost any task requires some degree of concentration and focus. Some non-limiting examples of such situations include responding to a series of email communications without becoming sidetracked by online or other distractions; doing homework; narrowly focusing on the mechanics of a physical activity, such as a team sport, cycling, weightlifting, and others, and increasing the efficiency of performing a variety of mental tasks associated with one's employment without becoming distracted.

In some embodiments, the mental condition is social anxiety. The mindfulness-increasing composition is provided for use to decrease social anxiety. Social anxiety is decreased by use of the mindfulness-increasing composition, in some embodiments, for persons who become anxious in a crowd of other people; in business meetings; at social events, including dates; and generally in situations wherein an anxious person becomes insecure. In some embodiments, the mindfulness-increasing composition optimizes social performance by allowing a person to freely exhibit the best qualities of their personality.

In some embodiments, the mental condition is decreased happiness or a depressed mood. The mindfulness-increasing composition is provided for use to increase the mood of a person. Some non-limiting examples wherein the mindfulness-increasing composition is provided for improving mood include situations wherein the user wishes to feel content, happy, or motivated. In some embodiments, providing the mindfulness-increasing composition allows the user to feel an increased degree of motivation to perform work-related physical and mental tasks. In some embodiments, provision of the mindfulness-increasing composition makes work-related tasks more pleasurable for the user, and makes rote or repetitive tasks seem more interesting.

The mindfulness-increasing composition is provided by providing step 320, in some embodiments, to prevent a mental condition. For example, in some embodiments, the onset of social-related stress or anxiety is prevented wherein the composition is provided at least two hours prior to a scheduled social engagement. In a second non-limiting example, work-related mental fatigue is prevented. A third example of providing the mindfulness-increasing composition to prevent a condition is wherein the composition is provided to a user to prevent or mitigate the effects of a “hangover” which may arise from excess alcohol consumption. An additional example is wherein providing the composition prevents inhibitions, such as prior to public speaking or a public performance.

A mindfulness-increasing composition, methods of formation, and a method of use have been described. Based upon known mechanisms for individual components of the composition, several combinations of these components, particularly phenibut, L-theanine, and 5-HTP act synergistically in ways specific to the composition of the composition to increase alertness while also increasing a sense of calm, focus, and reducing anxiety. The synergistic effects of compositions comprising phenibut and L-theanine, phenibut and 5-HTP, and L-theanine with 5-HTP to improve several mental conditions are also described. The mindfulness-increasing composition also comprises a mindfulness-supportive mixture of vitamins and other nutrients for optimization of cognitive and physical function, overall neurological function, immune system function and general overall well-being. An aqueous-based delivery vehicle comprising a sweetener, a flavor, and a preservative is included in the description. Additionally, methods of use are described, allowing for effective, safe, and convenient use of a mindfulness-increasing composition by the end-user. Disclosed is a composition comprising 4-amino-3-phenylbutyric acid and L-theonine.

In some embodiments, the composition additionally comprises caffeine. In some embodiments, the composition additionally comprises chamomile flower extract. In some embodiments, the composition additionally comprises lemon balm extract. In some embodiments, the composition additionally comprises a vitamin. In some embodiments, the vitamin is vitamin B3 as niacinamide. In some embodiments, the vitamin is vitamin B6 as pyridoxine hydrochloride. In some embodiments, the vitamin is cyanocobalamin. In some embodiments, the vitamin is vitamin E as alpha tocopherol acetate.

In some embodiments, the composition additionally comprises choline. In some embodiments, the composition additionally comprises inositol. In some embodiments, the composition additionally comprises hyaluronic acid.

Disclosed is a composition comprising 4-amino-3-phenylbutyric acid and 5-hydroxytryptophan. In some embodiments, the composition additionally comprises caffeine. In some embodiments, the composition additionally comprises chamomile flower extract. In some embodiments, the composition additionally comprises lemon balm extract. In some embodiments, the composition additionally comprises a vitamin. In some embodiments, the vitamin is vitamin B3 as niacinamide. In some embodiments, the vitamin is vitamin B6 as pyridoxine hydrochloride. In some embodiments, the vitamin is cyanocobalamin. In some embodiments, the vitamin is vitamin E as alpha tocopherol acetate. In some embodiments, the composition additionally comprises choline. In some embodiments, the composition additionally comprises inositol. In some embodiments, the composition additionally comprises hyaluronic acid.

Disclosed is a composition comprising L-theanine and 5-hydroxytryptophan. In some embodiments, the composition additionally comprises caffeine. In some embodiments, the composition additionally comprises chamomile flower extract. In some embodiments, the composition additionally comprises lemon balm extract. In some embodiments, the composition additionally comprises a vitamin. In some embodiments, the vitamin is vitamin B3 as niacinamide. In some embodiments, the vitamin is vitamin B6 as pyridoxine hydrochloride. In some embodiments, the vitamin is cyanocobalamin. In some embodiments, the vitamin is vitamin E as alpha tocopherol acetate. In some embodiments, the composition additionally comprises choline. In some embodiments, the composition additionally comprises inositol. In some embodiments, the composition additionally comprises hyaluronic acid.

Disclosed is a composition comprising a mixture of 4-amino-3-hydroxybutyric acid, L-theanine, and 5-hydroxytryptophan in a ratio of about thirteen (13) parts 4-amino-3-hydroxybutyric acid to about four and one half (4.5) parts L-theanine to about one part 5-hydroxytryptophan. In some embodiments, the mixture is dissolved in water at a total concentration of about twenty-three point five (23.5) parts by weight of mixture to one (1) part by weight of water to form an aqueous solution. In some embodiments, the composition additionally comprises caffeine. In some embodiments, the composition additionally comprises chamomile flower extract. In some embodiments, the composition additionally comprises lemon balm extract. In some embodiments, the composition additionally comprises a vitamin. In some embodiments, the vitamin is vitamin B3 as niacinamide. In some embodiments, the vitamin is vitamin B6 as pyridoxine hydrochloride. In some embodiments, the vitamin is cyanocobalamin. In some embodiments, the vitamin is vitamin E as alpha tocopherol acetate.

Disclosed is a serving of a mindfulness-increasing composition comprising 4-amino-3-phenylbutyric acid, about 1,000 mg.; L-theanine, about 335 mg.; lemon balm extract, about 124 mg.; inositol, about 115 mg.; choline, about 115 mg.; chamomile flower extract, about 100 mg.; 5-hydroxy-L-tryptohpan, about 7 mg.; caffeine, about 50 mg.; hyaluronic acid, about 10 mg.; d-alpha tocopherol acetate, about 15 international units (“IU”); niacinamide, about 100 mg.; pyridoxine hydrochloride, about 25 mg.; and cyanocobalamin, about 100 micrograms (“mcg.”) in an aqueous solution wherein the total volume of the serving is about 60 (sixty) milliliters. In some embodiments, the amount of 4-amino-3-phenylbutyric acid is between about 250 milligrams (“mg”) and about 1,500 mg. In some embodiments, the amount of 4-amino-3-phenylbutyric acid is between about 20 mg and about 300 mg. In some embodiments, the amount of 4-amino-3-phenylbutyric acid is between about 300 mg and about 350 mg. In some embodiments, the amount of 4-amino-3-phenylbutyric acid is between about 350 mg and about 400 mg. In some embodiments, the amount of 4-amino-3-phenylbutyric acid is between about 400 mg and about 450 mg. In some embodiments, the amount of 4-amino-3-phenylbutyric acid is between about 450 mg and about 500 mg. In some embodiments, the amount of 4-amino-3-phenylbutyric acid is between about 550 mg and about 600 mg. In some embodiments, the amount of 4-amino-3-phenylbutyric acid is between about 600 mg and about 650 mg. In some embodiments, the amount of 4-amino-3-phenylbutyric acid is between about 650 mg and about 700 mg. In some embodiments, the amount of 4-amino-3-phenylbutyric acid is between about 700 mg and about 750 mg. In some embodiments, the amount of 4-amino-3-phenylbutyric acid is between about 750 mg and about 800 mg. In some embodiments, the amount of 4-amino-3-phenylbutyric acid is between about 380 mg and about 850 mg. In some embodiments, the amount of 4-amino-3-phenylbutyric acid is between about 850 mg and about 900 mg. In some embodiments, the amount of 4-amino-3-phenylbutyric acid is between about 900 mg and about 950 mg. In some embodiments, the amount of 4-amino-3-phenylbutyric acid is between about 950 mg and about 1,000 mg. In some embodiments, the amount of 4-amino-3-phenylbutyric acid is between about 1,000 mg and about 1,100 mg. In some embodiments, the amount of 4-amino-3-phenylbutyric acid is between about 1,100 mg and about 1,200 mg. In some embodiments, the amount of 4-amino-3-phenylbutyric acid is between about 1,200 mg and about 1,300 mg. In some embodiments, the amount of 4-amino-3-phenylbutyric acid is between about 1,300 mg and about 1,400 mg. In some embodiments, the amount of 4-amino-3-phenylbutyric acid is between about 1,400 mg and about 1,500 mg. In some embodiments, the amount of 4-amino-3-phenylbutyric acid is greater than about 1,500 mg. In some embodiments, the amount of 4-amino-3-phenylbutyric acid is less than about 250 mg.

In some embodiments, the amount of L-theanine is between about 50 mg and about 400 mg. In some embodiments, the amount of L-theanine is between about 50 mg and about 100 mg. In some embodiments, the amount of L-theanine is between about 100 mg and about 150 mg. In some embodiments, the amount of L-theanine is between about 150 mg and about 200 mg. In some embodiments, the amount of L-theanine is between about 200 mg and about 250 mg. In some embodiments, the amount of L-theanine is between about 250 mg and about 300 mg. In some embodiments, the amount of L-theanine is between about 300 mg and about 350 mg. In some embodiments, the amount of L-theanine is between about 350 mg and about 400 mg. In some embodiments, the amount of L-theanine is greater than about 400 mg. In some embodiments, the amount of L-theanine is less than about 50 mg.

In some embodiments, the amount of 5-hydroxytryptophan is between about 50 mg and about 300 mg. In some embodiments, the amount of 5-hydroxytryptophan is between about 50 mg and about 100 mg. In some embodiments, the amount of 5-hydroxytryptophan is between about 150 mg and about 200 mg. In some embodiments, the amount of 5-hydroxytryptophan is between about 200 mg and about 250 mg. In some embodiments, the amount of 5-hydroxytryptophan is between about 250 mg and about 300 mg. In some embodiments, the amount of 5-hydroxytryptophan is greater than about 300 mg. In some embodiments, the amount of 5-hydroxytryptophan is less than about 50 mg.

In some embodiments, the amount of caffeine is between about 50 mg and about 400 mg. In some embodiments, the amount of caffeine is about 50 mg. In some embodiments, the amount of caffeine is between about 50 mg and about 100 mg. In some embodiments, the amount of caffeine is between about 100 mg and about 150 mg. In some embodiments, the amount of caffeine is between about 150 mg and about 200 mg. In some embodiments, the amount of caffeine is between about 200 mg and about 250 mg. In some embodiments, the amount of caffeine is between about 250 mg and about 300 mg. In some embodiments, the amount of caffeine is between about 300 mg and about 350 mg. In some embodiments, the amount of caffeine is between about 350 mg and about 400 mg. In some embodiments, the amount of caffeine is greater than about 400 mg. In some embodiments, the amount of caffeine is less than about 50 mg.

In some embodiments, and amount of chamomile flower extract is between about 400 mg. and about 1,600 mg. In some embodiments, and amount of chamomile flower extract is between about 400 mg. and about 500 mg. In some embodiments, and amount of chamomile flower extract is between about 500 mg. and about 600 mg. In some embodiments, and amount of chamomile flower extract is between about 600 mg. and about 700 mg. In some embodiments, and amount of chamomile flower extract is between about 700 mg. and about 800 mg. In some embodiments, and amount of chamomile flower extract is between about 800 mg. and about 900 mg. In some embodiments, and amount of chamomile flower extract is between about 900 mg. and about 1,000 mg. In some embodiments, and amount of chamomile flower extract is between about 1,000 mg. and about 1,100 mg. In some embodiments, and amount of chamomile flower extract is between about 1,100 mg. and about 1,200 mg. In some embodiments, and amount of chamomile flower extract is between about 1,200 mg. and about 1,300 mg. In some embodiments, and amount of chamomile flower extract is between about 1,300 mg. and about 1,400 mg. In some embodiments, and amount of chamomile flower extract is between about 1,400 mg. and about 1,500 mg. In some embodiments, and amount of chamomile flower extract is between about 1,500 mg. and about 1,600 mg. In some embodiments, and amount of chamomile flower extract is greater than about 1,600 mg. In some embodiments, the amount of chamomile flower extract is less than about 400 mg.

In some embodiments, the amount of lemon balm extract is between about 600 mg and about 1,600 mg. In some embodiments, and amount of lemon balm extract is between about 600 mg and about 700 mg. In some embodiments, and amount of lemon balm extract is between about 700 mg and about 800 mg. In some embodiments, and amount of lemon balm extract is between about 800 mg and about 900 mg. In some embodiments, and amount of lemon balm extract is between about 900 mg and about 1,000 mg. In some embodiments, and amount of lemon balm extract is between about 1,000 mg and about 1,100 mg. In some embodiments, and amount of lemon balm extract is between about 1,100 mg and about 1,200 mg. In some embodiments, and amount of lemon balm extract is between about 1,200 mg and about 1,300 mg. In some embodiments, and amount of lemon balm extract is between about 1,300 mg and about 1,400 mg. In some embodiments, and amount of lemon balm extract is between about 1,400 mg and about 1,500 mg. In some embodiments, and amount of lemon balm extract is between about 1,500 mg and about 1,600 mg. In some embodiments, and amount of lemon balm extract is greater than about 1,600 mg. In some embodiments, and amount of lemon balm extract is less than about 600 mg.

In some embodiments, the amount of pyridoxine hydrochloride is between about 5 mg and about 50 mg. In some embodiments, the amount of pyridoxine hydrochloride is between about 5 mg and about 10 mg. In some embodiments, the amount of pyridoxine hydrochloride is between about 10 mg and about 15 mg. In some embodiments, the amount of pyridoxine hydrochloride is between about 15 mg and about 20 mg. In some embodiments, the amount of pyridoxine hydrochloride is between about 20 mg and about 25 mg. In some embodiments, the amount of pyridoxine hydrochloride is between about 25 mg and about 30 mg. In some embodiments, the amount of pyridoxine hydrochloride is between about 30 mg and about 35 mg. In some embodiments, the amount of pyridoxine hydrochloride is between about 35 mg and about 40 mg. In some embodiments, the amount of pyridoxine hydrochloride is between about 40 mg and about 45 mg. In some embodiments, the amount of pyridoxine hydrochloride is between about 45 mg and about 50 mg. In some embodiments, the amount of pyridoxine hydrochloride is greater than about 50 mg. In some embodiments, the amount of pyridoxine hydrochloride is less than about 5 mg.

In some embodiments, the amount of cyanocobalamin is between about 0.05 mg and about 15 mg. In some embodiments, the amount of cyanocobalamin is between about 0.05 mg and about 0.15 mg. In some embodiments, the amount of cyanocobalamin is between about 0.15 mg and about 0.50 mg. In some embodiments, the amount of cyanocobalamin is between about 0.50 mg and about 1.0 mg. In some embodiments, the amount of cyanocobalamin is between about 1.0 mg and about 5.0 mg. In some embodiments, the amount of cyanocobalamin is between about 5.0 mg and about 10.0 mg. In some embodiments, the amount of cyanocobalamin is between about 10.0 mg and about 15.0 mg. In some embodiments, the amount of cyanocobalamin is greater than about 15.0 mg. In some embodiments, the amount of cyanocobalamin is less than about 0.05 mg.

In some embodiments, and amount of choline is between about 50 mg and about 500 mg. In some embodiments, and amount of choline is between about 50 mg and about 100 mg. In some embodiments, and amount of choline is between about 100 mg and about 150 mg. In some embodiments, and amount of choline is between about 150 mg and about 200 mg. In some embodiments, and amount of choline is between about 200 mg and about 250 mg. In some embodiments, and amount of choline is between about 250 mg and about 300 mg. In some embodiments, and amount of choline is between about 300 mg and about 350 mg. In some embodiments, and amount of choline is between about 350 mg and about 400 mg. In some embodiments, and amount of choline is between about 400 mg and about 450 mg. In some embodiments, and amount of choline is between about 450 mg and about 500 mg. In some embodiments, and amount of choline is greater than about 500 mg. In some embodiments, and amount of choline is less than about 50 mg.

In some embodiments, and amount of inositol is between about 50 and about 3000 mg. In some embodiments, and amount of inositol is between about 50 and about 100 mg. In some embodiments, and amount of inositol is between about 100 and about 150 mg. In some embodiments, and amount of inositol is between about 150 and about 200 mg. In some embodiments, and amount of inositol is between about 200 and about 250 mg. In some embodiments, and amount of inositol is between about 250 and about 300 mg. In some embodiments, and amount of inositol is between about 300 and about 400 mg. In some embodiments, and amount of inositol is between about 400 and about 500 mg. In some embodiments, and amount of inositol is between about 500 and about 600 mg. In some embodiments, and amount of inositol is between about 600 and about 800 mg. In some embodiments, and amount of inositol is between about 800 and about 1,000 mg. In some embodiments, and amount of inositol is between about 1,000 and about 1,500 mg. In some embodiments, and amount of inositol is between about 1,500 and about 2,000 mg. In some embodiments, and amount of inositol is between about 2,000 and about 3,000 mg. In some embodiments, and amount of inositol is greater than about 3,000 mg. In some embodiments, and amount of inositol is less than about 50 mg.

In some embodiments, the amount of hyaluronic acid is between about 5 mg. and about 200 mg. In some embodiments, the amount of hyaluronic acid is between about 25 mg. and about 50 mg. In some embodiments, the amount of hyaluronic acid is between about 50 mg. and about 100 mg. In some embodiments, the amount of hyaluronic acid is between about 100 mg. and about 200 mg. In some embodiments, the amount of hyaluronic acid is greater than about 200 mg. In some embodiments, the amount of hyaluronic acid is less than about 5 mg.

In some embodiments, the mindfulness-increasing composition additionally comprises a flavoring agent, sucralose, potassium sorbate; sodium benzoate; malic acid; citric acid; acesulfame potassium, and an artificial color.

Disclosed is a method of forming a mindfulness-increasing composition comprising a providing a mixture comprising 4-amino-3-phenylbutyric acid and L-theanine; and batching the mixture with ingredients of a mindfulness-increasing formulation into a solution. In some embodiments, the method further comprises a step packaging the mindfulness-increasing composition into a consumer unit.

Disclosed is a method of forming a mindfulness-increasing composition comprising a providing a mixture comprising L-theanine; and 5-hydroxytryptophan; and batching the mixture with ingredients of a mindfulness-increasing formulation into a solution. In some embodiments, the method further comprises a step packaging the mindfulness-increasing composition into a consumer unit.

Disclosed is a method of forming a mindfulness-increasing composition comprising a providing a mixture comprising 4-amino-3-phenylbutyric acid and 5-hydroxytryptophan; and batching the mixture with ingredients of a mindfulness-increasing formulation into a solution. In some embodiments, the method further comprises a step packaging the mindfulness-increasing composition into a consumer unit.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above.

What is claimed is:

1. A composition for increasing mindfulness comprising 4-amino-3-phenylbutyric acid and theanine.

2. The composition of claim 1, wherein the theanine comprises the L-enantiomer of theanine.

3. The composition of claim 1, wherein the theanine comprises a racemic mixture of theanine.

4. The composition of claim 1, additionally comprising an extract selected from the group of extracts consisting of chamomile flower extract and lemon balm extract.

5. The composition of claim 1, additionally comprising a vitamin selected from the group of vitamins consisting of vitamin B₃ as niacinamide, vitamin B₆ as pyridoxine hydrochloride, vitamin B₁₂ as cyanocobalamin and vitamin E as alpha tocopherol acetate.

6. The composition of claim 1, additionally comprising a substance selected from the group of substances consisting of choline, inositol, and hyaluronic acid.

7. The composition of claim 1, additionally comprising caffeine.

8. A composition for increasing mindfulness comprising 4-amino-3-phenylbutyric acid and 5-hydroxytryptophan.

9. The composition of claim 8, additionally comprising an extract selected from the group of extracts consisting of chamomile flower extract and lemon balm extract.

10. The composition of claim 8, additionally comprising a vitamin selected from the group of vitamins consisting of vitamin B₃ as niacinamide, vitamin B₆ as pyridoxine hydrochloride, vitamin B₁₂ as cyanocobalamin and vitamin E as alpha tocopherol acetate.

11. The composition of claim 8, additionally comprising a substance selected from the group of substances consisting of choline, inositol, and hyaluronic acid.

12. The composition of claim 8, additionally comprising caffeine.

13. A composition for increasing mindfulness and wellbeing comprising theanine and 5-hydroxytryptophan.

14. The composition of claim 13, additionally comprising an extract selected from the group of extracts consisting of chamomile flower extract and lemon balm extract.

15. The composition of claim 13, additionally comprising a vitamin selected from the group of vitamins consisting of vitamin B₃ as niacinamide, vitamin B₆ as pyridoxine hydrochloride, vitamin B₁₂ as cyanocobalamin and vitamin E as alpha tocopherol acetate.

16. The composition of claim 13, additionally comprising a substance selected from the group of substances consisting of choline, inositol, and hyaluronic acid.

17. The composition of claim 13, additionally comprising caffeine.

18. The composition of claim 13, additionally comprising 4-amino-3-phenylbutyric acid.

19. The composition of claim 13, wherein the theanine comprises the L-enantiomer of theanine.

20. The composition of claim 13, wherein the theanine comprises a racemic mixture of theanine.
